(19) United States
(12) Patent Application Publication
(10) Pub. No.: US 2022/0118158 A1
(43) Pub. Date: Apr. 21, 2022

(54) BIOPROSTHETIC GRAFT FOR CORONARY ARTERY BYPASS GRAFTING

(71) Applicant: enVVeno Medical Corporation, Irvine, CA (US)

(72) Inventor: Marc H. Glickman, Riverside, CA (US)

(21) Appl. No.: 17/507,607

(22) Filed: Oct. 21, 2021

Related U.S. Application Data
(60) Provisional application No. 63/094,873, filed on Oct. 21, 2020.

(51) Int. Cl. A61L 27/50; A61L 27/36; A61L 2/00

(52) U.S. Cl. CPC A61L 27/507; A61L 2/0035; A61L 27/3691; A61L 27/3625

(57) ABSTRACT

The present application provides a vascular bioprosthetic graft having an internal diameter of about 3 mm for performing coronary artery bypass grafting surgery. The present application also provides a method for manufacturing the vascular bioprosthetic graft. The vascular bioprosthetic graft is obtained from a xenogeneic venous vessel from the rectum, such as a xenogeneic cranio-rectal vein.

### CROSS-REFERENCE TO RELATED APPLICATIONS

This application claims priority to and the benefit of U.S. Provisional Patent Application No. 63/094,873, filed Oct. 21, 2020 which is herein incorporated by reference.

### FIELD

The present invention generally pertains to a vascular bioprosthetic graft for performing coronary artery bypass grafting surgery and a method for manufacturing the vascular bioprosthetic graft.

### BACKGROUND

Cardiovascular disease is one of the leading causes of death in the world. Narrowing of the coronary arteries through the deposition of cholesterol, lipids, and calcium in a process known as atherosclerosis, which is a primary factor in coronary occlusion of coronary artery disease. When percutaneous coronary intervention is deemed inappropriate for long-term revascularization in the condition of advanced atherosclerosis, a more invasive coronary artery bypass grafting (CABG) surgery may be performed. CABG is a type of surgery that improves blood flow to the heart by bypassing the blocked portion of a coronary vessel. Suitable bypass grafts for bypassing atherosclerotic coronary arteries can be harvested from the patient themselves (e.g., autografts), obtained from a donor (e.g., allografts), obtained from a different species (e.g., xenografts), obtained from synthetic vascular prostheses, or obtained from tissue-engineered vascular prostheses.

Autologous vessels, such as the saphenous vein, radial arteries and internal mammary thoracic artery harvested from the patient's body, are preferable grafts for small and medium-diameter vessels. However, these natural vessels have limited availability and invasive harvest is required. Due to repeat operations, graft failure or radiation thoracic treatment, the availability of autologous grafts can be limited. In particular, patients with serious comorbidities (e.g., end stage renal disease, diabetes or peripheral vascular disease) often have a lack of suitable autologous vessels for the bypass surgery.

Synthetic materials such as vascular grafts have been explored. However, there are concerns about using synthetic conduit with smaller diameters, since early graft occlusion is frequently encountered (Knight et al., Vascular grafting strategies in coronary intervention, Frontiers in Materials, Jun. 10, 2014, volume 1, article 4). Tissue-engineered vascular grafts which grow in vitro from vascular smooth muscle cells and then are decellularized, have also been explored. To date, small caliber prosthetic grafts have a very high incidence of thrombosis or occlusion. In general, various attempts to manufacture and use grafts for vascular repair have experienced significant failure rates because of undesirable dilation due to size mismatch between the graft and the host vessel, thrombosis, and/or complete endothelialization.

Many alternative conduits have been developed in the past for performing CABG surgeries when the availability of suitable autologous conduits is limited, but most of them have failed due to low patency rates or early thrombosis. As noted, small caliber prosthetic grafts have a very high incidence of thrombosis. It will be appreciated that a need exists for a vascular bioprosthetic graft for performing CABG surgery.

### SUMMARY

The present invention provides a vascular bioprosthetic graft having an internal diameter of about 3 mm for performing coronary artery bypass grafting surgery and a method for manufacturing the vascular bioprosthetic graft.

This disclosure provides a method of manufacturing a vascular bioprosthetic graft for a host vessel in a subject.

In one aspect, the method of manufacturing a vascular bioprosthetic graft for a host vessel in a subject comprises obtaining a xenogeneic venous vessel from dorsum; and subjecting the xenogeneic venous vessel to a fixation treatment; wherein the vascular bioprosthetic graft has an internal diameter of about 2.5-3.5 mm or preferably about 3 mm.

In some exemplary embodiments, the xenogeneic venous vessel from the rectum is a xenogeneic cranio-rectal vein. In some exemplary embodiments, the xenogeneic cranio-rectal vein is obtained from young bovine. In some specific exemplary embodiments, the age of the bovine is about 18 to about 24 months.

In an exemplary embodiment, the vascular bioprosthetic graft has an elastin to collagen ratio of about 8:1. In an exemplary embodiment, a normal saphenous vein has an elastin to collagen ratio of 2:1. This improved elastin to collagen ratio accounts for increase in improving retractile force and opposes the development of dilatation and tortuosity of the bioprosthetic graft.

In some exemplary embodiments, the vascular bioprosthetic graft is used as a bypass graft for conducting coronary artery bypass grafting surgery or coronary artery bypass procedures. In some aspects, the method of manufacturing a vascular bioprosthetic graft for a host vessel in a subject further comprises inserting a mandrel into an internal lumen of the xenogeneic venous vessel from the dorsum, wherein the mandrel has a diameter of about 3 mm.

In some exemplary embodiments, the method of the present application further comprises subjecting the vascular bioprosthetic graft to terminal gamma radiation sterilization.

In some exemplary embodiments, the subject in the method of the present application is human. In some exemplary embodiments, the host vessel in the method of the present application is a coronary artery. In some exemplary embodiments, the vascular bioprosthetic graft of the present application is capable of having substantial endothelialization after implementing the vascular bioprosthetic graft to the host vessel in a subject. In some exemplary embodiments, substantial endothelialization refers to about 70% endothelialization, about 75% endothelialization, about 80% endothelialization, about 85% endothelialization, about 90% endothelialization, about 95% endothelialization, about 96% endothelialization, about 97% endothelialization, about 98% endothelialization, about 99% endothelialization, or about 100% endothelialization.

Endothelialization is thought to be a critical step in establishing the long-term biocompatibility of cardiovascular devices. It has been observed that other grafts have not demonstrated the unique property of significant endothelialization after placement and is often viewed as important and difficult to achieve feature for prosthetic grafts as it has the potential to reduce thrombosis and development of intimal hyperplasia. However, the present invention shows partial to complete endothelialization after 90-180 days.

In some exemplary embodiments, the vascular bioprosthetic graft of the present application prevents thrombosis once placed in the host vessel.

In some exemplary embodiments, the vascular bioprosthetic graft of the present application prevents intimal hyperplasia once placed in the host vessel.

In some exemplary embodiments, the vascular bioprosthetic graft of the present application provides a substantial laminar flow once placed in the host vessel. In some exemplary embodiments, substantial laminar flow refers to a laminar flow of about 70-100%.

In some exemplary embodiments, the vascular bioprosthetic graft of the present application prevents eddy currents or turbulent flow once placed in the host vessel. In some exemplary embodiments, eddy currents or turbulent flow is reduced to about 70-100%.

In another aspect, the method of manufacturing a vascular bioprosthetic graft for a host vessel in a subject comprises obtaining a xenogeneic venous vessel from dorsum; and subjecting the xenogeneic venous vessel to a fixation treatment, wherein the fixation treatment comprises subjecting the vessel to a buffered isotonic solution; and subjecting the vessel to a glutaraldehyde solution. In some exemplary embodiments, the concentration of glutaraldehyde solution is about 0.2%. In some exemplary embodiments, the vessel is also subjected to a bioburden reduction treatment as well. This exemplary method decreases spoilage rate of the graft.

In some exemplary embodiments, the vascular bioprosthetic graft is used as a bypass graft for conducting coronary artery bypass grafting surgery or coronary artery bypass procedures. In some aspects, the method of manufacturing a vascular bioprosthetic graft for a host vessel in a subject further comprises inserting a mandrel into an internal lumen of the xenogeneic venous vessel from the dorsum, wherein the mandrel has a diameter of about 3 mm.

In some exemplary embodiments, the method of the present application further comprises subjecting the vascular bioprosthetic graft to terminal gamma radiation sterilization. In some aspects, the subject in the method of the present application is human. In some aspects, the host vessel in the method of the present application is a coronary artery. In some aspects, the vascular bioprosthetic graft of the present application is capable of having endothelialization after implementing the vascular bioprosthetic graft to the host vessel in a subject.

In yet another aspect, the method of manufacturing a vascular bioprosthetic graft for a host vessel in a subject comprises obtaining a xenogeneic venous vessel from dorsum; and subjecting the xenogeneic venous vessel to a fixation treatment, wherein the fixation treatment comprises subjecting the vessel to a buffered isotonic solution, glutaraldehyde solution, and bioburden reduction treatment; and wherein elastin to collagen ratio of the vessel is about 8:1. This ratio of about 8:1 is an improvement as opposed to normal saphenous vein ratio of elastin to collagen of about 2:1. In some exemplary embodiments, the concentration of glutaraldehyde solution is about 0.2% cross-linked with the buffered isotonic solution.

In some exemplary embodiments, the xenogeneic venous vessel from the rectum is a xenogeneic cranio-rectal vein. In some exemplary embodiments, the xenogeneic cranio-rectal vein is obtained from a bovine. In some specific exemplary embodiments, the age of the bovine is about 24 months, or about 18-24 months.

In some exemplary embodiments, the vascular bioprosthetic graft is used as a bypass graft for conducting coronary artery bypass grafting surgery or coronary artery bypass procedures. In some aspects, the method of manufacturing a vascular bioprosthetic graft for a host vessel in a subject further comprises inserting a mandrel into an internal lumen of the xenogeneic venous vessel from the dorsum, wherein the mandrel has a diameter of about 3 mm.

In some exemplary embodiments, the method of the present application further comprises subjecting the vascular bioprosthetic graft to terminal gamma radiation sterilization. In some aspects, the subject in the method of the present application is human. In some aspects, the host vessel in the method of the present application is a coronary artery. In some aspect, the vascular bioprosthetic graft of the present application is capable of having endothelialization after implementing the vascular bioprosthetic graft to the host vessel in a subject.

In yet another aspect, the method of manufacturing a vascular bioprosthetic graft for a host vessel in a subject comprises obtaining a xenogeneic venous vessel from dorsum; and subjecting the xenogeneic venous vessel to a fixation treatment; wherein the vessel is dissected using a method comprising a sharp dissection of the vein along the path of the rectum and sigmoid colon of the bovine.

In some exemplary embodiments, the xenogeneic venous vessel from the rectum is a xenogeneic cranio-rectal vein. In some exemplary embodiments, the xenogeneic cranio-rectal vein is obtained from a bovine. In some specific exemplary embodiments, the age of the bovine is about 24 months, or about 18-24 months.

In an exemplary embodiment, this bioprosthetic vein has an elastin to collagen ratio of 8:1. In an exemplary embodiment, a normal saphenous vein has an elastin to collagen ratio of 2:1.

In some exemplary embodiments, the vascular bioprosthetic graft is used as a bypass graft for conducting coronary artery bypass grafting surgery or coronary artery bypass procedures. In some aspects, the method of manufacturing a vascular bioprosthetic graft for a host vessel in a subject further comprises inserting a mandrel into an internal lumen of the xenogeneic venous vessel from the dorsum, wherein the mandrel has a diameter of about 3 mm.

In some exemplary embodiments, the method of the present application further comprises subjecting the vascular bioprosthetic graft to terminal gamma radiation sterilization. In some aspects, the subject in the method of the present application is human. In some aspects, the host vessel in the method of the present application is a coronary artery. In some aspect, the vascular bioprosthetic graft of the present application is capable of having endothelialization after implementing the vascular bioprosthetic graft to the host vessel in a subject.

These, and other, aspects of the invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the invention.

### DETAILED DESCRIPTION

The present application provides a vascular bioprosthetic graft for a vessel reconstruction procedure and a method for manufacturing the vascular bioprosthetic graft for the vessel reconstruction. In particular, the present application provides a vascular bioprosthetic graft for coronary artery bypass grafting (CABG) surgery. The vascular bioprosthetic graft of the present application has an internal diameter of about 3 mm and can be used as a conduit to bypass the blocked portion of the coronary artery to improve blood flow to the heart.

Cardiovascular disease, such as coronary artery disease, coronary heart disease, cerebrovascular disease, peripheral arterial disease, and deep vein thrombosis, is often associated with the narrowing or blockage of blood vessels leading to reduced blood flow and tissue damage due to inadequate nutrient supply. Vascular reconstruction may involve endovascular procedures, such as angioplasty, stent insertion, atherectomy to widen a stenosed vessel or remove the obstruction, or replacing or bypassing a damaged or occluded vessel using a vascular graft.

For patients with suitable veins, the current standard of care for most CABG surgeries is to harvest the saphenous vein from the leg of the patient, dissect the saphenous vein grafts into multiple grafts, and to use the dissected grafts to revascularize the heart. In addition to the vein harvest procedure being invasive, painful, and subject to its own complications for the patient, such grafts are also known to have high short term and long-term failure rates when used as grafts around the heart. Studies indicate that up to 40% of such grafts fail within one year of CABG surgeries, with a significant percentage failing within the first 30 days. Eight to ten years after surgery, SVG failure rates are known to be in as high as 75%.

The blood vessels have variable diameters, including larger-diameter vessels (e.g., greater than about 8 mm), such as aortoiliac replacements; medium-diameter vessels (e.g., about 6-8 mm), such as carotid or common femoral artery replacements; and small-diameter vessels (e.g., less than about 6 mm) such as the coronary arteries, infrainguinal arteries (below the inguinal ligament), and infrageniculate arteries (below the knee) (Pashneh-Tala et al., The Tissue-Engineered Vascular Graft—Past, Present, and Future, Tissue Engineering: Part B, Volume 22, Number 1, 2016).

Approximately 200,000 CABG surgeries are performed each year in the U.S., representing more than 55% of all cardiac surgeries and accounting for between $15 Billion and $25 Billion in annual expenditures.

Presently, there are no FDA-approved prosthetic grafts for coronary artery bypass procedures. ProCol® vascular bioprosthesis received FDA premarket approval for hemodialysis access, which is derived from bovine mesenteric vein with about 6 mm internal diameter. ProCol® vascular bioprosthesis was implanted for infrainguinal reconstruction which yielded a disappointing result of a primary patency rate of 0% (Kovalic et al.). There are long-felt unmet needs for providing a vascular graft which has a small diameter (e.g., less than about 6 millimeters) for coronary artery bypass procedures. The development of a small diameter vascular graft for coronary artery bypass procedures has been pursued. However, there is a lack of success in pursuing small-diameter prosthetic vascular grafts. In some instances, the results of these efforts have yielded devices with theoretical promise, but with little practical success, since there often are issues associated with small diameter conduits, such as thrombosis and kinking.

Vascular graft failures are commonly associated with thrombosis, intimal hyperplasia, atherosclerosis, or infection. Thrombosis occurs as a result of damage to endothelial cells lining of the graft lumen, leading to the adherence of blood proteins and the activation of clotting mechanisms. Intimal hyperplasia is caused by the migration of vascular smooth muscle cells from the vessel media to the intima and their proliferation and extracellular matrix deposition. Intimal hyperplasia may occur in the graft vessel or in the native vessel around the anastomosis due to multiple causes, including (i) compliance mismatch between the graft and native vessel; (ii) vessel diameter mismatch; (iii) damage to, or a lack of, endothelial cells; (iv) suture line stress concentrations; (v) trauma during surgery; and (vi) hemodynamic factors causing blood flow disturbances. Flow disturbances seems to be the leading cause for alterations in graft biology leading to neointimal formation and graft occlusion. Atheroma formation is associated with invading vessel neointima by monocytes forming macrophages to develop atherosclerotic plaque. Graft infection can be common in synthetic conduits due to their susceptibility to bacterial colonization. (Pashneh-Tala et al.) In addition, a variety of strategies have been proposed to utilize tissue-engineering approaches by enhancing the endothelialization of the tissue-engineered grafts.

The cellular and molecular recovery processes after coronary intervention include the involvement of vascular cells and progenitor cells for arterial remodeling, neointimal proliferation, and re-endothelialization. The involvement of bone marrow-derived progenitor cells are critical for vascular inflammation responses and vascular repairs. Endothelial progenitor cells can mobilize from bone marrow into peripheral blood to promote endothelial regeneration for post-natal neovascularization. In the condition of vascular injury, there is a balance between endothelial-like stem cell responses which favor re-endothelialization and smooth muscle-like stem cell responses which promote restenosis. (Inoue et al., Vascular inflammation and repair, implication for re-endothelialization, restenosis, and stent thrombosis, JACC: Cardiovascular Interventions, vol. 4, No. 10, 2011).

There are options for bypass grafts, such as autografts, allografts, xenografts, synthetic vascular prostheses, or tissue-engineered vascular prostheses. Selecting bypass grafts (e.g., conduits) to perform CABG surgery for treating coronary artery disease depends on a number of factors, such as location of the blockage, extent of the blockage, size of the coronary arteries, availability of arteries and veins, and patient medical factors. Autographs, such as autologous vascular grafts, are natural vessel grafts which are pieces of healthy blood vessels harvested from a patient's body, such as a vein from the patient's leg or an artery in the patient's chest. Natural blood vessels, such as saphenous vein, radial artery, internal mammary thoracic artery, or internal mammary artery, are commonly used as autologous bypass grafts. The saphenous vein is frequently used due to its technical ease of use, or in cases of multiple grafting procedures or those patients undergoing repeat surgeries (Knight et al.). However, harvesting blood vessels from the patient for performing CABG, the medical condition may create a morbidity in the patient resulting in poor wound healing and pain simultaneously in a patient with comorbid insomnia and anxiety. Furthermore, the long-term patency of the arterial or venous grafts have significant impact on the success of CABG surgery.

The left internal mammary artery (LIMA), an artery running inside the ribcage and close to the sternum, has been used to revascularize the left side of the heart, particularly to the left descending coronary artery (LAD). The use of LIMA graft has become the gold standard for re-vascularizing the left side of the heart during CABG surgery, since LIMA graft provides excellent short term and long-term patency. For the right side of the heart, and where additional grafts are needed on the left side, the current standard of care is to harvest the saphenous vein (SV) from the patient's leg to be dissected into pieces and used as bypass grafts around the heart. Unfortunately, SV grafts are not nearly as effective as the LIMA graft for re-vascularizing the heart, and have high short-term and long-term failure rates, for example, where the SV graft becomes blocked or occluded and thereby depriving the heart of necessary blood flow. For example, it has been reported that between ten to forty percent of SV grafts used as conduits for CABG surgeries fail within the first year after the CABG surgery. (Lee, Michael et. al., Saphenous Vein Graft Intervention, Journal of the American College of Cardiology: Cardiovascular Interventions Vol 4 No. 8 Aug. 2011: 831-43). A significant percentage of SV grafts fail within the first 30 days. At 10 years, it has been reported that the failure rates of the SV grafts can be as high as 75%. (Gaudino, Mario, et. al., The Choice of Conduits in Coronary Artery Bypass Surgery, Journal of the American College of Cardiology Vol 66 No. 15 Oct. 2015 1729-37). This failure is often primarily due to two main causes: endothelial damage of the transplanted graph during harvest; and size mismatch.

As noted above, the causes of SV graft failure can include size mismatch and a thickening of the interior of the SV graft that begins immediately following the harvest procedure known as the endothelial to mesenchymal transition. Size mismatch can occur since the diameter of SV graft is often significantly larger than the diameter of the coronary arteries around the heart. The problem of size mismatch leads to flow disturbances, since the flow rate of the blood flow can be variable while traveling through vessels with variable diameters, eventually leading to graft thromboses and graft failure. The thickening of the cell walls of SV graft also can occur when a layer of endothelial cells on the inner surface of the SV graft is disturbed at the beginning of the harvesting procedure by starting a chain reaction which causes the cells to thicken and the inside of the graft to narrow, subsequently resulting in blood clots and graft failure (Daylon, James, et. al., Maladapted Endothelial Cells Flip the Mesenchymal Switch; Science Translational Medicine 12 Mar. 2014: Vol. 6, Issue 227, pp. 227 fs12).

Xenograft, such as decellularized vascular grafts derived from animal tissues, are commercially available based on decellularized bovine blood vessels, such as Artegraft®, Solcograft®, and ProCol® (Pashneh-Tala et al.). ProCol® vascular bioprosthesis is derived from bovine mesenteric vein processed with glutaraldehyde, which has about 6 mm internal diameter with a usable length of about 10-40 cm. ProCol® vascular bioprosthesis is intended for the creation of a bridge graft for vascular access subsequent to at least one previously failed prosthetic access graft (ProCol® product information). ProCol® received FDA premarket approval approved for hemodialysis access. An investigation was performed by implanting ProCol® for infrainguinal reconstruction which yielded a disappointing result of a primary patency rate of 0% (Kovalic et al., Outcome of ProCol, a Bovine Mesenteric Vein Graft, in Infrainguinal Reconstruction, Eur J Vasc Endovasc Surg 24, 533-534 (2002), 10.1053/ejvs.2002.1710).

Synthetic materials as vascular grafts also have been explored. Poly(ethylene terephthalate) (PET®, Dacron), expanded poly(tetrafluoroethylene) (ePTFE, Gore-Tex®), and heparin-bonded ePTFE (Propaten®) are commercially available synthetic grafts. The synthetic vascular grafts have demonstrated some degree of satisfactory long-term results as vascular grafts for larger vessel diameters (e.g., greater than about 8 mm) and medium vessel diameters (e.g., about 6-8 mm). There are concerns of using synthetic conduit with smaller diameters (e.g., about 3-6 mm), since early graft occlusion is frequently encountered, resulting from thrombogenicity of the synthetic surface and anastomotic hyperplasia (Knight et al.). Although Dacron and ePTFE grafts have been used successfully in peripheral revascularization, the small-calibre vascular grafts made of synthetic materials have not been successful used for CABG due to low long-term patency rates. In addition, ePTFE has poor mechanical characteristics and lacks endothelial cells as lining of the lumen of the graft. These can be significant factors leading to poor patency. Dacron grafts suffer from thrombosis and neo-intimal proliferation. ePTFE grafts also have had poor patency rates because of surface thrombogenicity. (Desai et al., Role of prosthetic conduits in coronary artery bypass grafting, European Journal of Cardio-thoracic Surgery 40 (2011) 394-398). Tissue-engineered vascular grafts, such as HAV (Human Acellular Vessel) from Humacyte (Durham, N.C.), have also been explored. These grafts are vessels growing in vitro from vascular smooth muscle cells and then decellularized.

The present application provides a xenogeneic vascular bioprosthetic graft for CABG surgery, wherein the vascular bioprosthesis graft is derived from venous vessel from a dorsum of a bovine. In a preferred embodiment, a vein with a high elastin and collagen content from the dorsum of a bovine, is used to obtain the xenogeneic vascular bioprosthetic graft of the present application for CABG surgery. A normal saphenous vein has an elastin to collagen ratio of about 2:1. In some embodiments, the venous vessel from the dorsum, which has an internal diameter of about less than about 6 mm, such as about 3 mm preferably, with usable length of about 5-30 cm, such as about 20 cm preferably, is processed with a glutaraldehyde based fixation, In some preferred exemplary embodiments, the xenogeneic vascular bioprosthesis graft of the present application has an internal diameter of about 3 mm with usable length of about 20 cm, which is derived from a vein in the dorsum of a young bovine, of up to about 24 months in age.

The vascular bioprosthetic graft of the present application can be used as a conduit for CABG surgery to bypass the blocked portion of coronary arteries to improve blood flow to the heart. Studies of implanting the vascular bioprosthetic graft of the present application in sheep demonstrated complete endothelialization within 90 days or within 180 days. The vascular bioprosthetic graft of the present application has been demonstrated in pre-clinical study to sustain effective coronary hemodynamics and cardiac function. The vascular bioprosthetic graft of the present application fulfills the long-felt unmet needs by providing a vascular graft which has a small diameter (e.g., less than about 6 millimeters) for coronary artery bypass procedures. The vascular bioprosthetic graft of the present application provides the advantages of complete endothelialization of the grafts, such as providing a scaffolding for endothelial migration to accelerate vascular repair. In addition, since the vascular bioprosthetic graft of the present application has a small diameter, for example, about 3 mm, it provides the advantages of reducing the compliance mismatch between the graft and native vessel, such as reducing vessel diameter mismatch. Furthermore, the vascular bioprosthetic graft of the present application provides anatomic and functional characteristics to mimic the native vessels around the heart, which satisfy the demands of coronary artery blood flow.

The vascular bioprosthetic graft of the present application provides the advantages of allowing more complete revascularization of the diseased heart with the goal of eliminating the need for vein and artery harvesting procedures, thus reducing the comorbidities and complications attendant to graft harvesting. The advantages of the vascular bioprosthetic graft also include consistently providing sufficient uniform bypass grafts for comprehensive cardiac muscle revascularization, eliminating the related complications and chronic postoperative discomfort frequently reported for autologous graft harvest. In addition, the vascular bioprosthetic graft of the present application more closely matches the size of the arteries around the heart, eliminating graft failures that occur due to size mismatch, and can adapt to the prevailing haemodynamic conditions. Furthermore, the vascular bioprosthetic graft of the present application encourages and promotes more complete endothelialization which is critical to improve graft performance and long-term viability. The xenogeneic vascular bioprothetic graft also provides strength, viscoelasticity, biocompatibility, blood compatibility and biostability as alternative conduit for performing CABG surgeries when the availability of suitable autologous conduits is limited.

The vascular bioprosthetic graft of the present application can be beneficial to particular patient populations, such as patients who have experienced previous CABG surgery and need multiple vessel redo surgery; patients who do not have usable internal mammary artery due to previous surgery; patients who lost their saphenous vein to previous surgeries; patients who have had previous bilateral vein stripping (or ablation therapies) or harvested and require multiple bypass grafts; patients on chronic dialysis with previous CABG surgery and who do not have usable leg veins or internal mammary artery for CABG surgery, since hemodialysis radial arteries cannot be harvested; patients who have amputation of one or both legs and previous CABG surgery; and elderly female patients with diabetes and previous CABG surgery that require multiple vessel bypass surgeries. For most of these patients, only one internal mammary artery is usable, since the use of the second internal mammary artery is attended by a high risk of sternal complications. The vascular bioprosthetic graft of the present application also can be used with patients who present for either a first or redo CABG procedure with veins that are not suitable for harvesting due to previous saphenous vein thrombosis or trauma to the veins during harvest and patients who had previous CABG surgeries utilizing vein grafts and suffer serious accidents during coronary angioplasty or other coronary catheter procedures with the requirements of immediate multiple vessel bypass.

The limited availability of natural vessels and failures of vascular grafts have led to a demand for an effective vascular bioprosthetic graft with high patency rate-for performing coronary artery bypass grafting surgery. They also have led to a demand for methods for performing coronary artery bypass grafting surgery using the vascular bioprosthetic graft. This disclosure provides vascular bioprosthetic grafts and manufacturing methods to satisfy the aforementioned demands and the long-felt unmet needs for performing coronary artery bypass grafting surgery.

Exemplary embodiments disclosed herein satisfy the aforementioned demands and the long-felt needs by providing a vascular bioprosthetic graft having an internal diameter of about 3 mm for performing coronary artery bypass grafting surgery and a method for manufacturing the vascular bioprosthetic graft.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

As used herein, the terms “include,” “includes,” and “including,” are meant to be non-limiting and are understood to mean “comprise,” “comprises,” and “comprising,” respectively.

In some exemplary embodiments, the disclosure provides a method of manufacturing a vascular bioprosthetic graft for a host vessel in a subject, the method comprising: obtaining a xenogeneic venous vessel from the dorsum; and subjecting the xenogeneic venous vessel from the dorsum to a fixation treatment; wherein the vascular bioprosthetic graft has an internal diameter of about 2.5-3.5 mm or about 3 mm. In some exemplary embodiments, the xenogeneic venous vessel from the dorsum is a xenogeneic vein which is obtained from a bovine.

As used herein, the term “bioprosthetic graft” refers to a prosthetic graft consisting of an animal part or containing animal tissue. A “prosthetic graft” or “prosthesis” refers to an artificial object to replace a missing or impaired part of the body.

As used herein, the term “from the dorsum” includes at least the superior mesenteric vein and the inferior mesenteric vein toward the hemorroidal veins. It ends near pancreas and forms the hepatic portal vein by coming together with the splenic vein.

As used herein, the term “fixation treatment” is a treatment used to prevent or arrest the degenerative processes, which commence as soon as a tissue is deprived of its blood supply. During fixation treatment, substantial changes can occur to the composition and appearance of cell and tissue components. Thus, selecting the treatment is critical.

### Exemplary Embodiments

In some exemplary embodiments, this disclosure provides a method of manufacturing a vascular bioprosthetic graft for a host vessel in a subject, the method comprising: obtaining a xenogeneic venous vessel from the dorsum; and subjecting the xenogeneic venous vessel from the dorsum to a fixation treatment; wherein the vascular bioprosthetic graft has an internal diameter of about 2.5-3.5 mm or about 3 mm. In some exemplary embodiments, the xenogeneic venous vessel from the rectum is a xenogeneic vein which is obtained from a bovine, wherein the xenogeneic venous vessel from the dorsum is obtained from a bovine, wherein the age of the bovine is up to about 24 months of age.

In some exemplary embodiments, the vascular bioprosthetic graft of the present application has an internal diameter of about 2.8-3.5 mm, about 3-3.5 mm, about 3 mm, about less than 6 mm, about 2.8 mm, about 2.9 mm, about 3.1 mm, about 3.2 mm, about 3.3 mm, about 3.4 or about 3.5 mm.

In some exemplary embodiments, a usable length of the vascular bioprosthetic graft is about 5-30 cm, about 10-40 cm, about 10 cm, about 15 cm, about 16 cm, about 17 cm, about 18 cm, about 19 cm, about 20 cm, about 21 cm, about 22 cm, about 23 cm, about 24 cm, about 25 cm or about 30 cm.

In some exemplary embodiments, the age of the bovine is about 24 months, about 18-24 months, about 18 months, about 19 months, about 20 months, about 21 months, about 22 months, or about 23 months.

The consecutive labeling of method steps as provided herein with numbers and/or letters is not meant to limit the method or any embodiments thereof to the particular indicated order.

Various publications, including patents, patent applications, published patent applications, accession numbers, technical articles and scholarly articles are cited throughout the specification. Each of these cited references is incorporated by reference, in its entirety and for all purposes, herein.

The disclosure will be more fully understood by reference to the following Examples, which are provided to describe the disclosure in greater detail. They are intended to illustrate and should not be construed as limiting the scope of the disclosure.

### EXAMPLES

### Example 1. Preparation of Vascular Bioprosthetic Grafts for Coronary Artery Bypass Procedures

Bovine veins were collected for preparing vascular bioprosthetic grafts for coronary artery bypass procedures. The venous vessel from the dorsum was harvested from a bovine, such as a young steer. It is preferable that the age of the bovine is up to about 30 months of age. The bovine vessel was collected by carefully cutting through the vein and separating the vein away from the sigmoid colon to the rectum. The harvested vein was maintained at its natural condition without stretching. The collected bovine vein was cleaned by rinsing and washing the internal lumen of the vein with isotonic saline solution without damaging internal lining of the vein lumen. The veins were strengthened to relieve any kinked sections. The veins were trimmed to remove unacceptable sections, such as having cuts, tears or obvious deviation from size specification, such as internal diameters in the range of about 3-3.5 mm. The harvested vein was evaluated for containing competent valves. The collateral branches of the bovine vein were ligated under 2 mm of pressure with 6-0 surgical suture that has been approved to being sterilized with gamma radiation. The bioprosthetic device is terminally sterilized with 25 kGys gamma radiation.

A glass or quartz mandrel having a 3 mm diameter (e.g., a solid 3 mm diameter rod with polished ends) was obtained. The 3 mm mandrel was inserted gently into one end of the vein followed by continuously sliding the strengthened vein onto the mandrel without stretching the vein. This process of inserting a mandrel was used to ensure that the bovine vein was straightened with uniform internal diameter of about 3 mm. The vein should slip easily onto the mandrel without stretching or damaging the internal lumen of the vein. The veins have small internal diameters, which had to be stretched for slipping onto the mandrel, were discarded. Some tension existed when veins having proper diameters were slipped onto the mandrels. The veins having internal diameters that were too large for slipping onto the 3 mm mandrel were discarded. Subsequently, the veins were treated with glutaraldehyde solution for chemical crosslinking of the vein to derive the vascular bioprosthetic graft. The vascular bioprosthetic grafts of the present application were prepared in lengths up to 20 cm having internal diameters in the range of about 3-3.5 mm. The bioprosthetic graft was packed in a container with storage buffer, such as buffered physiological saline, with terminal gamma radiation sterilization.

The vascular bioprosthetic grafts were evaluated using pulsatile flow condition to simulate physiological pressure of blood flow. The shear stress of the grafts were evaluated. They were also evaluated for performing coronary artery bypass procedures in animals, such as sheep. The test results indicated that the vascular bioprosthetic graft having about 3 mm internal diameter was most desirable for performing coronary artery bypass procedures, since it mimics the average size of a coronary vessel.

### Example 2. Evaluation of the Vascular Bioprosthetic Grafts in Animal Models

The vascular bioprosthetic graft of the present application prepared in Example 1 was evaluated regarding mechanical properties and hemodynamics. The results indicated that this vessel had similar compliance as native vessels around the heart. The vascular bioprosthetic graft of the present application was used to perform coronary artery bypass grafting procedures in animal models, such as sheep. The bioprosthetic graft was provided in about 20 cm length having about 3 mm internal diameter and stored sterile in buffered physiological saline with terminal gamma radiation sterilization. The results indicated that no intimal hyperplasia was seen at either the donor LAD vessel or within the graft at 90 days and 180 days after the bypass grafting procedure. There was no evidence of graft degeneration. In addition, endothelialization of the grafts were seen both at 90 and 180 days. In animal studies performed at the American Preclinical Services in Coon Rapids, Mn., the bioprosthetic device was implanted into sheep. The bypass was performed from the aorta to the left descending coronary vessel (LAD) in sheep. Necropsy was performed at 60, 90 and 180 days. At 90 days, a thin layer of endothelial cells were seen throughout the entire graft and no evidence of intimal hyperplasia was noted. At 60 days, partial endotheliazation was noted but complete was seen at 90 days.

What is claimed is:

1. A method of manufacturing a vascular bioprosthetic graft for a host vessel in a subject, the method comprising:
obtaining a xenogeneic venous vessel from dorsum of a bovine; and
subjecting the xenogeneic venous vessel to a fixation treatment to obtain a bioprosthetic graft;
wherein the vascular bioprosthetic graft has an internal diameter of about 2.5-3.5 mm or
about 3 mm, wherein said graft provides a substantial laminar flow once placed in the host vessel.

2. The method of claim 1, wherein the xenogeneic venous vessel from the dorsum is a xenogeneic vein.

3. The method of claim 1, wherein the xenogeneic venous vessel from the dorsum is a xenogeneic cranio-rectal vein.

4. The method of claim 1, wherein the xenogeneic venous vessel has an elastin to collagen ratio of about 8:1.

5. The method of claim 1, wherein the xenogeneic venous vessel from the dorsum is obtained from a bovine, wherein the age of the bovine is up to 24 months old.

6. The method of claim 1, wherein the vascular bioprosthetic graft is used as a bypass graft for conducting coronary artery bypass grafting surgery or coronary artery bypass procedures.

7. The method of claim 1, further comprising inserting a mandrel into an internal lumen of the xenogeneic venous vessel from the dorsum.

8. The method of claim 7, wherein the mandrel has a diameter of about 3 mm.

9. The method of claim 1, wherein size of the graft matches the diameter of the host vessel.

10. The method of claim 1, wherein the graft prevents thrombosis once placed in the host vessel.

11. The method of claim 1, wherein the graft prevents intimal hyperplasia once placed in the host vessel.

12. The method of claim 1, wherein the graft substantially prevents eddy currents or turbulent flow once placed in the host vessel.

13. The method of claim 1, wherein the graft provides substantially similar pulsability as the host vessel.

14. The vascular bioprosthetic device of claim 1, wherein the graft is capable of having substantial endothelialization after implementing the vascular bioprosthetic graft to the host vessel in a subject.

15. The method of claim 1, further comprising subjecting the vascular bioprosthetic graft to terminal gamma radiation sterilization.

16. The method of claim 1, wherein the subject is human.

17. The method of claim 1, wherein the host vessel is a coronary artery.

18. The method of claim 1, wherein the vascular bioprosthetic graft is capable of having substantial endothelialization after implementing the vascular bioprosthetic graft to the host vessel in a subject.

19. A vascular bioprosthetic device for a host vessel in a subject, comprising a vascular bioprosthetic graft manufactured by the method of claim 1.

20. The vascular bioprosthetic device of claim 19, wherein the xenogeneic venous vessel from the dorsum is a xenogeneic cranio-rectal vein obtained from a bovine, wherein the age of the bovine is about 24 months, or about 18-24 months.

21. The vascular bioprosthetic device of claim 19, wherein the vascular bioprosthetic graft is used as a bypass graft for conducting coronary artery bypass grafting surgery or coronary artery bypass procedures.

22. The vascular bioprosthetic device of claim 19, wherein the vascular bioprosthetic graft is capable of having endothelialization after implementing the vascular bioprosthetic graft to the host vessel in a subject.

23. A method for coronary artery bypass grafting surgery in a subject using a vascular bioprosthetic graft, the method comprising:
obtaining a xenogeneic venous vessel from dorsum of a bovine;
subjecting the xenogeneic venous vessel to a fixation treatment to obtain a bioprosthetic graft; and
attaching the graft in a coronary artery of said subject,
wherein the vascular bioprosthetic graft has an internal diameter of about 2.5-3.5 mm or about 3 mm and wherein said graft is capable of having substantial endothelialization after implementing the vascular bioprosthetic graft to the host vessel in a subject.

24. The method of claim 23, wherein the xenogeneic venous vessel from the dorsum is a xenogeneic vein.

25. The method of claim 23, wherein the xenogeneic venous vessel from the dorsum is a xenogeneic cranio-rectal vein.

26. The method of claim 23, wherein the xenogeneic venous vessel has an elastin to collagen ratio of about 8:1.

27. The method of claim 23, wherein the xenogeneic venous vessel from the dorsum is obtained from a bovine, wherein the age of the bovine is up to 24 months old.

28. The method of claim 23, wherein size of the graft matches the diameter of the host vessel.

29. The method of claim 23, wherein the graft prevents thrombosis once placed in the host vessel.

30. The method of claim 23, wherein the graft prevents intimal hyperplasia once placed in the host vessel.

31. The method of claim 23, wherein the graft substantially prevents eddy currents or turbulent flow once placed in the host vessel.

32. The method of claim 23, wherein the graft provides substantially similar pulsability as the host vessel.

33. The method of claim 23, further comprising subjecting the vascular bioprosthetic graft to terminal gamma radiation sterilization.

34. The method of claim 23, wherein the ubject is human.
